(19) United States
(12) Patent Application Publication
(10) Pub. No.: US 2022/0378771 A1
(43) Pub. Date: Dec. 1, 2022

(54) MODIFYING THE EXPRESSION LEVEL OF A GENE ENCODING AN CYCLOOXYGENASE ENZYME BY TREATING A HUMAN SUBJECT WITH A NITROXIDE

(71) Applicant: Louis HABASH, Irvine, CA (US)

(72) Inventor: Louis HABASH, Irvine, CA (US)

(21) Appl. No.: 17/330,131

(22) Filed: May 25, 2021

(51) Int. Cl. A61K 31/45; A61P 39/06

(52) U.S. Cl. CPC A61K 31/45; A61P 39/06

(57) ABSTRACT

Some embodiments disclosed herein include a method for increasing an expression level of a gene. The methods can include identifying a human subject having an increased expression level of COX1; and administering to the human subject an effective amount of a nitroxide antioxidant, whereby expression level of the gene is decreased.

### BACKGROUND

### Field

The present disclosure relates generally to the field of modulation of gene expression and more particularly to decreasing expression levels of one or more genes relating to cyclooxygenase 1 by treating human subjects with a nitroxide.

### Description of the Related Art

Diseases and conditions are treatable by adjusting the expression levels and activities of key genes in the body. Gene expression irregularities, whether overexpressed, activated, under expressed or inhibited underlie the development and progression of every disease and condition. Some diseases are characterized by deficient expression of certain genes while other diseases result from over expression of certain genes. A disease resulting from irregular gene expression can be prevented, treated, or reversed by administering a nitroxide antioxidant to target and correct the expression levels of the genes.

Expression levels of genes are often naturally controlled in an appropriate way, but sometimes natural control of gene expression fails. For example, in cancer, genes providing instructions for cell growth are activated or switched on, when they should be off. Autoimmune diseases and aging are other examples of diseases and conditions that result from irregular gene expression. As cells age, the natural control of gene expression deteriorates promoting several diseases and conditions such as inflammation, chronic pain, infections, neurodegenerative disease, neurological disorders, skin diseases, etc. It is essential to identify the irregular expression of the genes involved in the cause of the disease and adjust the expression levels of those genes.

Often referred to as gene therapy, the targeting and correction of cellular dysfunction through adjusting the expression level of certain genes is necessary to prevent, treat, or reverse a disease or condition. Only by identifying key genes and developing therapeutics that altering the expression patterns of those genes can we prevent the development of the disease, reduce its effects once it has occurred, or reverse it all together.

One of the key genes involved in several diseases and conditions is cyclooxygenase 1 (COX1). When this gene is overexpressed it causes several diseases and conditions associated with the overexpression of the gene. Thus, correction of the overexpression of COX1 genes is essential for treatment and prevention of the associated diseases and conditions.

### SUMMARY

Some embodiments disclosed herein provide methods for increasing gene expression. The methods, in some embodiments, include identifying a human subject over the age of 35 and having an increased expression level of COX1; and administering to the human subject an effective amount of a nitroxide antioxidant resulting in a decreased expression level of the gene. In some embodiments, the gene is COX1. In some embodiments, the human subject is aging. In some embodiments, the human subject is over the age of 55. In some embodiments, the human subject is over the age of 65. In some embodiments, the expression level of the gene in a skin tissue is decreased by treatment. In some embodiments, the expression level of the gene in an adipose tissue is decreased by treatment. In some embodiments, the expression level of the gene in blood is decreased by treatment. In some embodiments, the expression level of the gene in a neuronal tissue is decreased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for increasing the expression level of a gene in a human subject in need thereof, comprising: identifying a human subject having an increased expression level of COX1; administering to the human subject an effective amount of a nitroxide antioxidant, whereby the expression level of COX1 is decreased. In some embodiments, the gene is COX1. In some embodiments, the decreased expression level of the gene is age-related. In some embodiments, the human subject is over the age of 35. In some embodiments, the human subject is over the age of 45. In some embodiments, the human subject is over the age of 55. In some embodiments, the human subject is over the age of 65. In some embodiments, the decreased expression level of the gene is disease-related. In some embodiments, the disease is selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the disease is age-related. In some embodiments, the expression level of the gene in a skin tissue is decreased by treatment. In some embodiments, the expression level of the gene in an adipose tissue is decreased by treatment. In some embodiments, the expression level of the gene in blood is decreased by treatment. In some embodiments, the expression level of the gene in a neuronal tissue is decreased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for reducing risk of a disease in a human subject in need thereof, comprising: identifying a human subject over the age of 35 having an decreased risk of a disease due to an increased expression level of COX1; administering to the human subject an effective amount of a nitroxide antioxidant, whereby the expression level of COX1 is decreased. In some embodiments, the disease is selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the gene is COX1. In some embodiments, the human subject is over the age of 45. In some embodiments, the human subject is over the age of 55. In some embodiments, the human subject is over the age of 65. In some embodiments, the expression level of the gene in a skin tissue is decreased by treatment. In some embodiments, the expression level of the gene in an adipose tissue is decreased by treatment. In some embodiments, the expression level of the gene in blood is decreased by treatment. In some embodiments, the expression level of the gene in a neuronal tissue is decreased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods comprising: identifying a human subject having or at risk of developing a cancer and in need of a decreased expression level of a COX1 gene ; administering to the human subject an effective amount of a nitroxide antioxidant, whereby the expression level of the gene associated with cyclooxygenase enzyme and cyclooxygenase activity is decreased. In some embodiments, the cancer can be selected from the group consisting of bladder cancer, colorectal cancer, hepatocellular carcinoma, prostate carcinoma, and kidney carcinoma. In some embodiments, the gene is COX1. In some embodiments, the cancer is age-related. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is decreased by treatment. In some embodiments, the expression level of the gene in an adipose tissue is decreased by treatment. In some embodiments, the expression level of the gene in blood is decreased by treatment. In some embodiments, the expression level of the gene in a neuronal tissue is decreased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods comprising: identifying a human subject having or at risk of developing an autoimmune disease and in need of an decreased expression level of a COX1 gene; administering to the human subject an effective amount of a nitroxide antioxidant, wherein the expression level of the gene associated with cyclooxygenase enzyme and cyclooxygenase activity is decreased. In some embodiments, the autoimmune disease can be selected from the group consisting of rheumatoid/osteoid arthritis, systemic lupus erythematosus (SLE), inflammatory bowel disease, multiple sclerosis, atherosclerosis, and osteoporosis. In some embodiments, the gene is COX1. In some embodiments, the autoimmune disease is age-related. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is decreased by treatment. In some embodiments, the expression level of the gene in an adipose tissue is decreased by treatment. In some embodiments, the expression level of the gene in blood is decreased by treatment. In some embodiments, the expression level of the gene in a neuronal tissue is decreased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for a disease associated with an increased expression level of cyclooxygenase enzyme and cyclooxygenase activity is decreased in a patient in need thereof, comprising: identifying a human subject having or at risk of developing a disease associated with an increased expression of COX1; administering to the human subject an effective amount of a nitroxide antioxidant, whereby the expression level of COX1 is decreased. In some embodiments, the disease can be selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the gene is COX1. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is decreased by treatment. In some embodiments, the expression level of the gene in an adipose tissue is decreased by treatment. In some embodiments, the expression level of the gene in blood is decreased by treatment. In some embodiments, the expression level of the gene in a neuronal tissue is decreased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for treating an individual in need thereof, comprising: identifying an individual over the age of 35 in need of an decreased expression level of COX1; and administering to the individual an effective amount of a nitroxide antioxidant to increase the level of expression of the gene associated with cyclooxygenase enzyme and cyclooxygenase activity. In some embodiments, the gene is COX1. In some embodiments, the human subject is over the age of 45. In some embodiments, the human subject is over the age of 55. In some embodiments, the human subject is over the age of 65. In some embodiments, the human subject has an decreased expression level of the gene. In some embodiments, the individual has or is at risk of developing an age-related condition. In some embodiments, the age-related condition comprises decreased senescence in a tissue. In some embodiments, the age-related condition comprises inhibition cyclooxygenase enzyme and cyclooxygenase activity in a tissue. In some embodiments, the age-related condition comprises decreased molecular heterogeneity. In some embodiments, the age-related condition comprises decreased functional impairment in a tissue. In some embodiments, the expression level of the gene in a skin tissue is decreased by treatment. In some embodiments, the expression level of the gene in an adipose tissue is decreased by treatment. In some embodiments, the expression level of the gene in blood is decreased by treatment. In some embodiments, the expression level of the gene in a neuronal tissue is decreased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for treating an individual in need thereof, comprising: identifying an individual having a disease-related increased expression level of COX1; and administering to the individual an effective amount of a nitroxide antioxidant to increase the level of expression of the gene associated with cyclooxygenase enzyme and cyclooxygenase activity. In some embodiments, the disease can be selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the gene is COX1. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is decreased by treatment. In some embodiments, the expression level of the gene in an adipose tissue is decreased by treatment. In some embodiments, the expression level of the gene in blood is decreased by treatment. In some embodiments, the expression level of the gene in a neuronal tissue is decreased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for treating an individual having or at risk of developing a condition due to aging, comprising: identifying an individual over the age of 35; and administering to the individual an effective amount of a nitroxide antioxidant, whereby the expression level of the gene associated with cyclooxygenase enzyme and cyclooxygenase activity is decreased. In some embodiments, the individual has an decreased expression level of the gene. In some embodiments, the gene is COX1. In some embodiments, the condition is an age-related condition. In some embodiments, the age-related condition comprises decreased senescence in a tissue. In some embodiments, the age-related condition comprises overactivation of COX1 in a tissue. In some embodiments, the age-related condition comprises decreased molecular heterogeneity. In some embodiments, the age-related condition comprises decreased functional impairment in a tissue. In some embodiments, the age-related condition is selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the human subject is over the age of 35. In some embodiments, the human subject is over the age of 45. In some embodiments, the human subject is over the age of 55. In some embodiments, the human subject is over the age of 65.

Some embodiments disclosed herein provide methods for increasing the expression level of a gene in a human subject in need thereof, comprising: identifying a human subject having an increased expression level of COX1; and delivering to the human subject an effective amount of a nitroxide antioxidant to increase the level of expression of the gene associated with cyclooxygenase enzyme and cyclooxygenase activity. In some embodiments, the nitroxide antioxidant is 4-hydroxy-2,2,6,6-tetramethylpiperidine-1-oxyl. In some embodiments, the decreased expression level of the gene is age-related. In some embodiments, wherein the decreased expression level of the gene is cancer-related. In some embodiments, the decreased expression level of the gene is disease-related. In some embodiments, the decreased expression level of the gene is neurodegeneration-related. In some embodiments, the decreased expression level of the gene is infection related. In some embodiments, the decreased the level of expression of the gene improves cyclooxygenase activity and prostaglandin formation. In some embodiments, the expression level of the gene is decreased in a tissue selected from the group consisting of a skin tissue, an immune tissue, an adipose tissue, a pancreatic tissue, cardiac tissue, and a neuronal tissue by treatment.

Some embodiments disclosed herein provide methods for increasing an expression level, in an eukaryotic cell, of one or more genes encoding cyclooxygenase proteins involved in arachidonic acid metabolism by contacting the eukaryotic cell with a nitroxide antioxidant. In some embodiments, the one or more genes is COX1. In some embodiments, the nitroxide antioxidant is 4-hydroxy-2,2,6,6-tetramethylpiperidine-1-oxyl. In some embodiments, the eukaryotic cell is a cancer cell. In some embodiments, the expression level of the one or more genes is decreased in said cell in a tissue selected from the group consisting of a skin tissue, an immune tissue, an adipose tissue, a pancreatic tissue, cardiac tissue, and a neuronal tissue. In some embodiments, prior to said contacting, the eukaryotic cell exhibits an age-related decreased expression level of said one or more genes. In some embodiments, prior to said contacting, the eukaryotic cell exhibits a disease-related decreased expression level of said one or more genes. In some embodiments, prior to said contacting, the eukaryotic cell exhibits a neurodegeneration-related expression level of said one or more genes.

Some embodiments disclosed herein provide methods for improving chemotherapeutic response in a human subject comprising: contacting cancer cells in the subject with an effective amount of a nitroxide antioxidant whereby a level of expression of cyclooxygenase enzyme and cyclooxygenase activity is decreased in said cancer cells. In some embodiments, said cancer cells are known to have increased COX1 function. In some embodiments, the decreased expression level of one or more genes following treatment initiates apoptosis within one or more of said cancer cells. In some embodiments, the decreased expression level reduces or prevents resistance to other chemotherapeutic agents. In some embodiments, the nitroxide antioxidant is 4-hydroxy-2,2,6,6-tetramethylpiperidine-1-oxyl. In some embodiments, the gene is selected from the group consisting of COX1.

Some embodiments disclosed herein provide methods for increasing cyclooxygenase enzyme and cyclooxygenase activity in a human subject comprising: identifying a human subject known to have increased COX1 function ; and delivering to the subject an effective amount of a nitroxide antioxidant, whereby a level of cyclooxygenase enzyme and cyclooxygenase activity is decreased. In some embodiments, the nitroxide antioxidant is 4-hydroxy-2,2,6,6-tetramethylpiperidine-1-oxyl. In some embodiments, increased COX1 function is age-related. In some embodiments, the increased COX1 function is cancer-related. In some embodiments, the increased COX1 function is disease-related. In some embodiments, the increased COX1 function is neurodegeneration-related. In some embodiments, the increased COX1 function is infection-related. In some embodiments, the decreased level of expression of the gene improves remodeling of damaged tissues. In some embodiments, the expression level of the gene is decreased in a tissue selected from the group consisting of a skin tissue, an immune tissue, an adipose tissue, a pancreatic tissue, cardiac tissue, and a neuronal tissue following treatment.

Some embodiments disclosed herein provide methods for treating a human subject having cancer comprising: delivering an effective amount of a nitroxide antioxidant to a human subject, wherein the human subject has previously been administered at least one chemotherapeutic agent, whereby a level of expression of cyclooxygenase enzyme and cyclooxygenase activity is decreased. In some embodiments, the human subject having cancer is identified with an increased expression of COX1. In some embodiments, the methods further comprise administering a promotor of a COX1 to the human subject.

### DETAILED DESCRIPTION

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

All patents, applications, published applications and other publications referred to herein are incorporated by reference for the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

As used herein, the term “expression” means the detection of a gene product that is expressed or produced by a nucleic acid molecule by standard molecular biology methods, which gene product refers to e.g. an unspliced RNA, an mRNA, a splice variant mRNA, a polypeptide, a post-translationally modified polypeptide, a splice variant polypeptide etc., and specifically products made using an RNA gene product as a template, e.g. cDNA of the RNA.

As used herein, “differential expression” of a gene means that the expression of the gene is at a higher level (“decreased expression”) or lower level (“decreased expression”) in a human subject suffering from a disease, for example cancers and autoimmune diseases, relative to its expression in a normal or control subject. Differential expression includes both quantitative, as well as qualitative, differences in the temporal or cellular expression pattern in a gene or its expression products among, for example, normal and diseased cells, or among cells which have undergone different disease events or disease stages.

As used herein, “increasing the expression level” of a gene means causing the expression of the gene to decrease by treating the human subject with a compound, for example a nitroxide antioxidant, such that the expression level of the gene after treatment is lower than the expression level of the gene before treatment in the human subject.

As used herein, “delivering” a compound shall mean bringing that compound into contact with a relevant cell, tissue, or organism. Similarly, “contacting” shall mean that the compound contacts a relevant target, such as a tissue or cell or tumor. In either case, delivery or contact in an organism can be affected by directly administering the compound to the organism, or by administering a different compound to the organism, such as a prodrug that is converted in vivo to the desired compound. In short, these terms cover any action that leads to contact between the desired compound and a target cell, tissue, or organism.

The present disclosure describes methods of modulating gene expression in human subjects. However, this is illustrative only and not intended to be limiting. For example, the methods disclosed herein can be used for modulating gene expression in other vertebrates, such as but not limited to mammals, birds, reptiles, fish, and the like (with modifications wherein appropriate). Mammals and birds include most agricultural animals. Treatment of companion animals, e.g., dogs, cats, or birds is also contemplated.

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### Human Subject Identification

The present disclosure relates to methods of treating alteration in gene expression, such as age-related, cancer-related, disease-related, neurodegeneration-related, and infection-related alteration in gene expression. Gene expression changes also play important roles in aging and serve as biomarkers of physiological decline and disease conditions, such as neurodegenerative diseases, and cancers. Therefore, one aspect of the present disclosure is methods of treating a human subject having an age-related, cancer-related, disease-related, neurodegeneration related, and/or infection-related decrease in gene expression levels, such as those genes associated with cyclooxygenase enzyme and cyclooxygenase activity. In some embodiments, the human subject can be identified based on the human subject's age, gene expression level, family history, health conditions, medical history, habits, or a combination thereof.

Regardless of the cause of the upregulation, some common terminology can be used. In some embodiments, the expression level of a gene (e.g., COX1) in a human subject is considered to be upregulated or increased if the increase in the expression level of that gene is statistically significant compared to that of a control or a reference. In some embodiments, the expression level of a gene (e.g., COX1) in a human subject is considered to be upregulated or increased if the increase in the expression level of that gene is statistically significant compared to that of a control or a reference. The control or reference can be, for example, a normal healthy population, a population at large, a collection of individuals of the same age or condition or sex, or the same human subject at a different time (e.g., at an earlier time of life when the human subject does or does not have the disease or condition that results in the upregulation).

In some embodiments, a normal healthy population or a population at large can be a population having the same or similar gender, age, and/or race, compared to the human subject. In some embodiments, the expression level of the gene in the control or reference can be the mean or median expression level of the gene in control subjects in the control or reference subjects in the reference. The increase in expression level can be statistically significant if the probability of the observed difference occurring not by chance, the confidence level, is greater than a threshold. The threshold can be, or be about, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or a number or a range between any two of these values.

In some embodiments, the increase in expression level can be, or be about, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, or a number or a range between any two of these values. In some embodiments, the increase in expression level can be at least 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, or more.

In some embodiments, the human subject may have an age that is, is about, or is over 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old.

In some embodiments, the human subject is identified based on the human subject's expression profiles of COX1. Non-limiting exemplary methods for determining the human subject's expression profiles include: amplification techniques such as PCR and RT-PCR (including quantitative variants), hybridization techniques such as in situ hybridization, microarrays, blots, and others, and high throughput sequencing techniques like Next Generation Sequencing (Illumina, Roche Sequencer, Life Technologies SOLID™), Single Molecule Real Time Sequencing (Pacific Biosciences), True Single Molecule Sequencing (Helicos), or sequencing methods using no light emitting technologies but other physical methods to detect the sequencing reaction or the sequencing product, like Ion Torrent (Life Technologies). Non-limiting exemplary methods for determining the human subject's expression profiles include: binding techniques such as ELISA, immunohistochemistry, microarray and functional techniques such as enzymatic assays.

### Targeted Gene Expression Adjustment

All living organisms are comprised of cells that function individually as well as in combination with other cells to form larger and more complex structures such as tissue and organs. The operation of each cell is based on the genetic instructions provided by the DNA contained therein. DNA is arranged in a particular sequence referred to as a gene which is transcribed and translated into a functional product required for the operation of the cell.

Genes are expressed in a particular quantity based on the instruction provided by the DNA. In particular, gene expression describes transcription of gene encoding DNA sequences into complementary DNA (cDNA) and translation of cDNA into the functional products, such as proteins. Many factors, both internal and external, are involved in regulation of gene expression in cells. Such regulation can manifest in an adjustment of gene expression to increase or decrease a number of proteins made.

The quantity of expression for a particular gene or group of complementary genes can be considered relative to a healthy state or disease state of the cell. In a healthy state, genes are expressed in a predictable quantity necessary for the operation of the cell. In a disease state, the genes are overexpressed or under expressed relative to the healthy-state expression. The deviation from the healthy state of gene expression may result in catastrophic burden on the cell due to over or under production of the functional product encoded by the gene.

A condition or disease may be identified based on such dysfunctional expression of genes within the cell. Whether the dysfunctional expression of the genes is due external influence on the cell or genetic aberrations, correction to the dysfunctional expression is necessary to address the underlying cause of the condition or disease. Overexpression or under expression of a gene or genes often results in dysfunction of downstream actions controlled by the same. Whether the gene is a regulator of cellular function or a vital in a responsive mechanism, modulation of the gene expression is a fundamental directive in addressing the foundational issues associated with many diseases and conditions. Differences often exist in therapy directives. Treatments for a disease or condition may be directed at addressing a manifestation or symptom of the disease. However, the underlying disease may be permitted to remain resulting in subsequent presentation of the previously treated symptoms. Therefore, it is essential to correct or reinforce the underlying cause of the disease. Ultimately, the treatment of the disease or condition requires targeting and modulating the expression level of the gene or genes that are overexpressed or under expressed.

Cyclooxygenase enzymes are responsible for the production of prostaglandins. Prostaglandins are involved in several different biological processes from homeostatic function to acute response to external stimuli.

In a healthy state, cyclooxygenase enzymes, such as COX1 and COX2 are responsible for the regulated production of the prostaglandins for healthy cellular function. However, overexpression or elevated levels of the enzymes or their encoding genes is associated with numerous diseases and conditions. The elevated levels or inducement of the cyclooxygenase enzymes may preempt the disease or condition, thereby causing the disease state; or the overexpression and elevated levels may be responsive and result in poor prognosis or promotion of the disease or condition. Whether the inducement or overexpression of these enzymes is reactive, or due to a genetic abnormality, it is the dysfunctional expression that defines a disease state for the subject.

Certain conditions, such as cardiovascular diseases, diabetes, obesity, and aging can be associated with (e.g., causes or caused by) overexpression of cyclooxygenase encoding genes resulting in the disfunction of vital cellular processes within cells and tissues. Thus, modulation of overexpressed and elevated quantity of cyclooxygenase enzymes or their encoding genes is essential for treatment and prevention of certain conditions.

Genes Associated with cyclooxygenase enzyme and cyclooxygenase activity

In some embodiments, administering to the human subject an effective amount of the nitroxide antioxidant results in a decreased expression level of a gene, for example COX1. Therefore, some embodiments disclosed herein provide methods for treating an individual in need thereof, comprising identifying an individual having a disease-related increased expression level of COX1; and administering to the individual an effective amount of a nitroxide antioxidant to decrease the level of expression of COX1. Some embodiments disclosed herein provide methods for treating an individual in need thereof, comprising identifying an individual in need of a decreased expression level of a COX1 gene; and administering to the individual an effective amount of a nitroxide antioxidant to decrease the level of expression of COX1. Some embodiments disclosed herein provide methods for treating an individual in need thereof, comprising: administering to the individual, known to have a disease-related increased expression level of COX1, an effective amount of a nitroxide antioxidant to decrease the level of expression of COX1. Some embodiments disclosed herein provide methods for treating an individual in need thereof, comprising: administering to an individual, known to be in need of an decreased expression level of a COX1 gene, an effective amount of a nitroxide antioxidant to increase the level of expression of cyclooxygenase enzyme and cyclooxygenase activity.

Non-limiting examples of diseases associated with altered level of cyclooxygenase enzyme and cyclooxygenase activity include cancer; breast cancer; lung cancer; kidney cancer; cancers of the ovary and uterus; cancer of the central nervous system; cancers of the head and neck; melanoma; lymphomas; leukemia; neurological disorders; Alzheimer's disease; Parkinson's disease; Huntington's disease; amyotrophic lateral sclerosis; stroke; cardiovascular disorders; ischemia; heart failure; infections, infectious diseases; bacterial infections; inflammatory responses; viral infections; autoimmune diseases; systemic lupus erythematosus; autoimmune lymphoproliferative syndrome; rheumatoid arthritis; and thyroiditis.

The gene associated with cyclooxygenase 1 can be COX1. For example, the treatment can result in decreased expression levels of COX1. The an decreased expression level of COX1, can decrease cyclooxygenase activity and prostaglandin formation. The decreased level of COX1 can result in a decrease in or disappearance of signs and symptoms of a disease associated with increased COX1 function, including the curing of the disease associated with increased COX1 function. In some embodiments, the decreased expression level of COX1, can decrease the level of cyclooxygenase enzyme and cyclooxygenase activity. The decreased level of cyclooxygenase enzyme and cyclooxygenase activity can result in a decrease in or disappearance of signs and symptoms of the disease associated with increased COX1 function , including the curing of the disease associated with increased COX1 function. In some embodiments, the decreased level of cyclooxygenase enzyme and cyclooxygenase activity can inhibit, suppress, prevent, or reverse the disease or the symptoms associated with the disease.

### Arachidonic Acid

Arachidonic acid is a polyunsaturated fatty acid covalently bound in esterified form in the cell membranes of most body cells. Following irritation or injury, arachidonic acid is released and oxygenated by enzyme systems leading to the formation of proinflammatory eicosanoids such as prostaglandins and related compounds. Prostaglandins and other prostanoids, products of the cyclooxygenase enzyme pathway, have potent inflammatory properties.

Arachidonic acid can be metabolized via enzymatic reactions involving cyclooxygenase 1, along with downstream enzymes that mediate the production of prostaglandins (PGH2, an unstable intermediate, PGE2, PGD2 and PGF2alpha, prostacyclins (PGI2), and thromboxanes (TXA2, TXB2). (Hanna, Violette Said, and Ebtisam Abdel Aziz Hafez. “Synopsis of arachidonic acid metabolism: A review.” Journal of advanced research vol. 11 23-32. 13 Mar. 2018, doi:10.1016/j.jare.2018.03.005; the content of which is incorporated herein by reference in its entirety).

It is generally accepted that arachidonic acid-derived prostaglandins not only contribute to the development of inflammation as intercellular pro-inflammatory mediators, but also promote the excitability of the peripheral somatosensory system, contributing to pain exacerbation. (Jang, Y., Kim, M. & Hwang, S. W. Molecular mechanisms underlying the actions of arachidonic acid-derived prostaglandins on peripheral nociception. J Neuroinflammation 17, 30 (2020); the content of which is incorporated herein by reference in its entirety). Metabolites of the enzymatic reaction between COX1 and arachidonic acid amplify the inflammatory signals to recruit leukocytes, pro-inflammatory cytokines, and immune cells to help in pathogens resistance and clearance. Second, they balance the induced inflammatory signals by producing resolving metabolites to act as host protection, since inflammation exerts a threatening action if it is not controlled in an appropriate time manner. COX-1 induced eicosanoids were shown to be lethal when produced continuously. Von Moltke et al. was able to prove that eicosanoids can be lethal, as COX1-mediated prostaglandins generation were shown to be responsible for vascular leakage and mortality in mice model. (Hanna, 2018).

Elevated levels of arachidonic acid have been associated with increases in inflammation. For example, rapid inflammation development via increased inflammatory enzyme expression, elevated lipid peroxidation product content and oxidative system impairment was caused by the high fat diet, which showed increases in arachidonic acid, as well as other polyunsaturated fatty acids (PUFAs). Changes in arachidonic acid content therefore, may be an early indicator of inflammation and irreversible changes in non-alcoholic fatty liver disease progression. Concurrent increases in arachidonic acid and COX1 result in elevated levels of proinflammatory metabolite prostaglandins.

### Cyclooxygenase 1

Cyclooxygenase 1, also known as prostaglandin G/H synthase 1, prostaglandin-endoperoxide synthase 1 (PTGS1) or prostaglandin H2 synthase 1, is an enzyme that in humans is encoded by the PTGS1 gene. COX1 is constitutively expressed in all tissues that induces an acute inflammation in response to short exposure to lipopolysaccharide (LPS) stimulation and also directs the cell to promote or suppress the leukotrienes (LTs) biosynthesis. COX1 prefers coupling and co-localization at perinuclear membrane or ER, with thromboxane synthase, prostaglandin F synthase, and two other prostaglandin D synthases isozymes, generating thromboxane A2 (TXA2), prostaglandin F2alpha, and prostaglandin D2, respectively. (Hanna, 2018).

The prostaglandins resulting from COX1 activity are known be proinflammatory and are associated with the cause or several disease states. Studies have shown that inhibition of COX reduces production of these inflammatory prostaglandins, thereby reducing inflammation and reducing the negative biological impact they are associated with.

### Prostaglandins: Thromboxane PGE, PGD, PGF, and PGI

Thromboxane (TXA) is a known metabolite of the reaction between COX1 and arachidonic acid. Increased COX1 results in increased levels of TXA. TXA has been associated with several disease states. For example, TXA induces hepatic damage through vasoconstriction, platelet aggregation, in-duction of leukocyte adhesion, up-regulation of proinflammatory cytokines, and induction of other vasoconstrictor release. In this regard, administration of cyclooxygenase inhibitor, specific thromboxane synthase inhibitor, and specific thromboxane receptor antagonists has been shown to protect from severe hepatic injury elicited by these hepatic stresses. (Yokoyama Y, Nimura Y, Nagino M, Bland K I, Chaudry I H. Role of Thromboxane in Producing Hepatic Injury During Hepatic Stress. Arch Surg. 2005;140(8):801-807. doi:10.1001/archsurg.140.8.801; the content of which is incorporated herein by reference in its entirety).

Thromboxane A2 (TXA2) plays a pivotal role in hepatic injury after ischemia-reperfusion.46 Numerous animal studies have shown that COX-1 inhibitors, COX-2 inhibitors, selective TXA2 synthase inhibitors, and specific TXA2 receptor antagonists protect the liver from injury after ischemia-reperfusion. These treatments reduce the damage to sinusoidal lining cells, ameliorate histological liver necrosis, lower the levels of serum transaminase, normalize amino acid metabolism and lipid peroxidation, and restore hepatic tissue blood flow. In hepatic cirrhosis, increased resistance in the portal venous system is the primary factor in the pathophysiology of portal hypertension. Increased resistance in the portal venous system is induced by the destruction of normal hepatic structure as a result of fibrin deposition or by the impaired balance between the action of vasodilators and vasoconstrictors. (Yokoyama, 2005).

Prostaglandin-endoperoxide synthases (PGEs) have been shown to be upregulated in several different disease states. In particular, PGEs are upregulated in various cancers. PGEs are a product of COX reaction with arachidonic acid and the increase of COX is directly related to an increase in PGE levels. COX1/2, PGs, and PG-endoperoxide synthases are connected to various pathological processes. Studies have shown that COX inhibitors and PGE2 receptor antagonist AH-23848B suppressed tumor growth in a xenograft derived from OE33 cells. (Tianshun Zhang, Qiushi Wang, Wei-Ya Ma, Keke Wang, Xiaoyu Chang, Michele L. Johnson, Ruihua Bai, Ann M. Bode, Nathan R. Foster, Gary W. Falk, Paul J. Limburg, Prasad G. Iyer, Zigang Dong. “Targeting the COX1/2-Driven thromboxane A2 pathway suppresses Barrett's esophagus and esophageal adenocarcinoma development”, EBioMedicine, November 2019; the content of which is incorporated herein by reference in its entirety).

COX-derived prostanoids include PGE₂, PGD₂, PGF_{2α}, PGI₂ (prostacyclin), and thromboxane A2, all of which can exert receptor-mediated effects in the airways and/or vasculature of the lung. For example, PGE2 and PGI2 possess bronchodilatory activity, whereas PGD₂ and PGF_{2α} are bronchoconstrictive. As such, COX products have received considerable attention regarding their potential roles in various lung diseases, with a particular emphasis on asthma and allergic inflammation. In humans, inhaled PGE2 inhibits the early- and late-phase pulmonary responses to inhaled allergen and attenuates aspirin- and exercise-induced bronchoconstriction in susceptible patients. (Cyclooxygenase-1 Overexpression Decreases Basal Airway Responsiveness but Not Allergic Inflammation. Jeffrey W. Card, Michelle A. Carey, J. Alyce Bradbury, Joan P. Graves, Fred B. Lih, Michael P. Moorman, Daniel L. Morgan, Laura M. DeGraff, Yun Zhao, Julie F. Foley and Darryl C. Zeldin. J Immunol Oct. 1, 2006, 177 (7) 4785-4793.

### Diabetes

Type II diabetes is a metabolic syndrome with increased glucose levels, abdominal obesity, high cholesterol and blood pressure. These conditions create stress in the biological system due to inflammation and ROS generation. It was suggested that poor glycemic control in subjects increases ROS production which leads to stimulation of the redox pathway. ROS is managed by enzymatic and non-enzymatic mechanisms. Different antioxidant enzymes viz. glutathione S-transferase, superoxide dismutase, catalase and COX maintain homeostasis in the cell. As mentioned earlier, COX are key regulators in the conversion of arachidonic acid into prostanoids which mediate inflammation, immunomodulation, apoptosis and blood flow. (Sushma Verma, Honey Chandra, Monisha Banerjee, Cyclooxygenase 1 (COX1) expression in Type 2 diabetes mellitus: A preliminary study from north India, Egyptian Journal of Medical Human Genetics, Volume 17, Issue 1, 2016, Pages 41-45, ISSN 1110-8630; the content of which is incorporated herein by reference in its entirety).

It is a rate limiting enzyme in the arachidonic to prostanoid pathway. COX1 is expressed in inflammatory cells and restricts the macrophage platelet aggregation. It also leads to vasoconstriction and vasodilation with the help of angiotensin II derived reactive oxygen species. Sometimes in the case of high ROS production, it exerts vascular damage by initiating the redox signaling pathway. The expression of COX1 gene may be induced and increased by proinflammatory stimuli viz growth factors, cytokines and mitogens in various cells. However, some workers suggested that it is unaffected by cytokines and inflammatory molecules. The COX1 expression level increases at the time of onset of diabetes and is associated with excess cardiovascular morbidity. (Sushma, 2016).

Studies have shown that COX1 is increased in early stages of neuropathy. In particular, constitutively expressed COX1 is important for the development of neuropathic pain. Spinal COX1 expression increases early in experimental neuropathy. Data show that inhibition of COX1 during early stages prevents the development of two typical symptoms of painful neuropathies: allodynia, which describes a state of increased pain sensation in response to stimuli that are usually not sensed as painful, such as light touch; and hyperalgesia, which is an increased sensitivity to noxious stimuli. (Hanns Ulrich Zeilhofer, Kay Brune; A Role for Cyclooxygenase-1 in Neuropathic Pain?. Anesthesiology 2003; 99:1043-1044; the content of which is incorporated herein by reference in its entirety).

### Cancer

Cancer may originate in the chronic inflammation setting associated with persistent infections, immune-mediated damage, or prolonged exposure to irritants. On the other hand, the genetic and epigenetic alterations underlying the cancerogenesis process inevitably modify the tissue homeostasis and may induce a chronic inflammatory response. Irrespective of the presumed primary or secondary nature of the process, inflammatory cells and mediators can be detected in most tumor tissues, where they act on both tumor and stromal cells and contribute to determine a tumor-promoting microenvironment. (Pannunzio, Alessandra, and Mauro Coluccia. “Cyclooxygenase-1 (COX-1) and COX-1 Inhibitors in Cancer: A Review of Oncology and Medicinal Chemistry Literature.” Pharmaceuticals (Basel, Switzerland) vol. 11,4 101. 11 Oct. 2018, doi:10.3390/ph11040101; the content of which is incorporated herein by reference in its entirety).

In the general complexity of the inflammatory response (hundreds of chemical mediators have been identified, but how they function in a coordinated manner is still not fully understood), the arachidonic acid metabolites play a relevant role which appears intertwined with their functions in cell and tissue homeostasis. Prostaglandins, including PGD2, PGE2, PGF2a, PGI2 and thromboxane are produced from arachidonic acid by sequential actions of cyclooxygenases (COX1) and specific synthases, and they exert their effects in autocrine and/or paracrine manner mainly through G protein-coupled receptors (GPCRs) at the cell surface. (Pannunzio, 2018).

The involvement of prostaglandins in cancer was first evidenced in human esophageal carcinoma cells, when their invasive and metastatic potential in nude mice was found to be related to PGE2 and PGF2a production. Elevated levels of PGE2 have been found in numerous cancers, and its effects on multiple cell signaling pathways involved in tumor malignant phenotype induction and maintenance have been thereafter demonstrated. However, PGE2 is not the only PG involved in carcinogenesis. PGD2 potentially contributes to the colon cancer risk in ulcerative colitis; more recently, TXA2 was found to be involved in colorectal cancer pathophysiology, as well as in multiple myeloma and lung cancer cell proliferation. (Pannunzio, 2018).

COX1 overexpression also promotes tumor growth through several different mechanisms. For example, in in ovarian cancer, elevated levels of COX1 was found to promote angiogenesis. Angiogenesis, defined as the generation of new capillaries from preexisting vessels, is a critical factor in the sustained growth of solid tumors. Ovarian cancer is known to be highly vascular and is a primary cancer in which current antiangiogenic therapies are being tested. VEGF and the VEGF receptor flk-1 are highly expressed in a majority of ovarian epithelial tumors, and VEGF expression is a negative prognostic factor for the disease. The transcription factor HIF-la is a dominant regulator of VEGF gene transcription and induces significant increases in VEGF mRNA copy number in response to various stimuli by binding to a hypoxia-responsive element within the VEGF promoter. There is also a strong link between the COX pathway and angiogenesis. Data from multiple groups suggest that a major mechanism by which COX-derived PGs promote polyp growth in the colon is through the stimulation of new blood vessel growth. To determine whether regions within ovarian tumors demonstrating high COX-1 expression correlate with foci of prominent angiogenic activity, in situ hybridization was done probing for COX-1, VEGF, Flk-1, and HIF-1α in serial sections. Regions of ovarian epithelial cells exhibiting high COX-1 also expressed significant levels of HIF-1α and VEGF. High levels of flk-1 were seen in the endothelial cells located in the stroma adjacent to ovarian epithelial cells expressing COX-1, HIF-1α, and VEGF. The above data suggest that elevations in COX-1 expression are enhanced in regions of ovarian epithelial tumors undergoing extensive angiogenesis. (Cyclooxygenase-1 is Overexpressed and Promotes Angiogenic Growth Factor Production in Ovarian Cancer. Rajnish A. Gupta, Lovella V. Tejada, Beverly J. Tong, Sanjoy K. Das, Jason D. Morrow, Sudhansu K. Dey and Raymond N. DuBois. Cancer Res Mar. 1, 2003 (63) (5) 906-911; the content of which is incorporated herein by reference in its entirety).

Further studies have shown that COX1 over-expression in tumors and strong association of COX1 with multiple pro-tumorigenic pathways in ovarian cancer cells. (Wilson, Andrew J et al. “Aberrant over-expression of COX-1 intersects multiple pro-tumorigenic pathways in high-grade serous ovarian cancer.” Oncotarget vol. 6,25 (2015): 21353-68. doi:10.18632/oncotarget.3860 the content of which is incorporated herein by reference in its entirety). COX1 has been shown to be a potential molecular target in ovarian cancer. COX1 and COX2 are rate-limiting enzymes in the early steps of prostaglandin (PG) biosynthesis and convert the fatty acid arachidonic acid to biologically active PGs and thromboxane A2. Pro-tumorigenic functions of COX-generated PGs include increased tumor cell growth, avoidance of apoptosis, angiogenesis, epithelial-mesenchymal transition (EMT), and promotion of an endothelial immune barrier preventing cytotoxic T cell infiltration into tumors. While older understanding of COX1 suggested it as a constitutive ubiquitous “housekeeping” enzyme associated with physiologic PGs, and COX2 as an inducible enzyme whose over-expression is linked to production of pathophysiological PGs and cancer. It has since been found that COX1, rarely COX2, is over-expressed in multiple human and mouse models of ovarian cancer. Further, a potential pro-tumorigenic role for COX1 in ovarian cancer is inferred by the ability of COX1 inhibitors to suppress ovarian tumorigenesis in these models. (Wilson, 2015).

### Neurodegenerative Disease

Neurodegenerative diseases such as Alzheimer's Disease (AD) have complex pathogenesis. Neuroinflammation leading to neuronal dysfunction and cell death is generally a downstream impact of a genetic aberration, cellular dysfunction, and a build-up of cholesterol and placques. COX1 has been shown to be elevated in AD patients and associated with production of proinflammatory metabolites that cause or promote the progression of AD. For example, it is known that COX1 plays a role in excitotoxicity, resulting in significant neuron cell death. (Cyclo-oxygenase-1 and -2 differently contribute to prostaglandin E2 synthesis and lipid peroxidation after in vivo activation of N-methyl-D-aspartate receptors in rat hippocampus. Pepicelli O, Fedele E, Berardi M, Raiteri M, Levi G, Greco A, Ajmone-Cat M A, Minghetti L. J Neurochem. 2005 June; 93(6):1561-7; the content of which is incorporated herein by reference in its entirety).

Inhibition of COX1 reduces this neuroinflammation and prevents, delays, and treats AD. Studies show that non selective COX inhibitors such as indomethacin and diclofenac showed preferential inhibition of COX1 and positive clinical findings in AD trials. (Frautschy, Sally A. “Thinking outside the box about COX-1 in Alzheimer's disease.” Neurobiology of disease vol. 38,3 (2010): 492-4. doi:10.1016/j.nbd.2010.02.009; the content of which is incorporated herein by reference in its entirety).

Elevated COX1 expression has also been found in microglial cells during amyloid plaque progression. Amyloid plaque is a known contributor to the onset and progression of AD. The role of COX1 in neuroinflammation is also supported by the facts that COX-1 inhibition using drug or gene deletion reduces neuronal damage and inflammatory responses after injection of lipopolysaccharide or AP in mouse brain. In a study of APP-Tg mice, (S)-11C-KTP-Me demonstrated age-dependent changes that closely corresponded to the increase in amyloid plaques as revealed by immunohistochemistry with an Aβ1-16 antibody. Importantly, expression of COX1 was observed in activated microglia, which were tightly surrounded and enclosed by amyloid plaques. It has also been shown that the expression level of COX1 was constitutive and was not changed even in the lipopolysaccharide-induced inflamed area of the brain with activated microglia. Considering these results from our study, (S)-11C-KTP-Me could detect the increased numbers of activated microglia that is closely associated with development of Aβ plaques in AD progression. Ultimately, AD patients showed an increase in COX1—expressing microglia surrounding Aβ plaques. (Detection of Cyclooxygenase-1 in Activated Microglia During Amyloid Plaque Progression: PET Studies in Alzheimer's Disease Model Mice, Miho Shukuri, Aya Mawatari, Masahiro Ohno, Masaaki Suzuki, Hisashi Doi, Yasuyoshi Watana be, HirotakaOnoe, Journal of Nuclear Medicine February 2016, 57 (2) 291-296; DOI: 10.2967/jnumed.115.166116; the content of which is incorporated herein by reference in its entirety).

Further studies show that cyclooxygenase inhibition on multiple neuronal pathways that counteract the neurotoxic effects of early accumulating amyloid-β oligomers. (Nathaniel S. Woodling, Damien Colas, Qian Wang, Paras Minhas, Maharshi Panchal, Xibin Liang, Siddhita D. Mhatre, Holden Brown, Novie Ko, Irene Zagol-Ikapitte, Marieke van der Hart, Taline V. Khroyan, Bayarsaikhan Chuluun, Prachi G. Priyam, Ginger L. Milne, Arash Rassoulpour, Olivier Boutaud, Amy B. Manning-Bog, H. Craig Heller, Katrin I. Andreasson, Cyclooxygenase inhibition targets neurons to prevent early behavioural decline in Alzheimer's disease model mice, Brain, Volume 139, Issue 7, July 2016, Pages 2063-2081; the content of which is incorporated herein by reference in its entirety). These findings coincide with COX1 inhibition as a target for preventing clinical manifestations of AD, such as behavioral decline.

### Hepatic Damage and Disease

COX1 and associated downstream metabolites have been instigated in the development and progression of several different diseases and conditions relating to the liver. Hepatic damage, for example, can be induced by hepatic stress and is mediated by various mediators, including vasoactive agents, proinflammatory cytokines, reactive oxygen species, and eicosanoids. The action of these mediators results in microcirculatory dysfunction, leukocyte infiltration, damage of cellular membranes, development of fibrosis, and stasis of biliary flow. (Yokoyama, 2005).

Hepatic injury after hepatic stress is caused by several mechanisms, including inflammatory reaction and microcirculatory disturbance. Levels of thromboxane, a vasoconstrictive eicosanoid, have been shown to increase in systemic circulation after different types of hepatic stress such as endotoxemia, hepatic ischemia-reperfusion, hepatectomy, liver transplantation, hemorrhagic shock and resuscitation, hepatic cirrhosis, and alcoholic liver injury. The production of thromboxane from the liver is also enhanced under these stresses, which may act on the liver in an autocrine or a paracrine fashion. Kupffer cells, resident hepatic macrophages, may be a major source of stress-induced thromboxane, although other cell types in the liver such as sinusoidal endothelial cells and hepatocytes may also produce this eicosanoid. Thromboxane induces hepatic damage through vasoconstriction, platelet aggregation, induction of leukocyte adhesion, up-regulation of proinflammatory cytokines, and induction of other vasoconstrictor release. (Yokoyama, 2005).

Alcohol-related liver disease and damage is also mediated by the arachidonic acid pathway. In the presences of ethanol, PUFAs lead to liver damage. Previous studies have shown that rats given poly-unsaturated fatty acids with ethanol develop pathological liver injury, whereas none of the histological features of alcoholic liver injury develop in rats given saturated fat with ethanol. The polyunsaturated fatty acid important to the pathogenesis of liver injury in CE-treated rats is linoleic acid. Therefore, the metabolism of linoleic acid to arachidonic acid and its subsequent conversion to eicosanoids is one possible mechanism involved in alcohol-induced liver injury. (Thromboxane Inhibitors Attenuate Pathological Changes in Alcoholic Liver Disease in the Rat. AMIN A. NANJI, SHAMSUDDIN KHWAJA, AMIR RAHEMTULLA, LILI MIAO, SHUPING ZHAO, and STEVEN R. TAHAN GASTROENTEROLOGY 1997;112:200-207; the content of which is incorporated herein by reference in its entirety).

### COX1 Inhibition After Physical Injury

Inhibition of COX1 is beneficial for healing after a physical injury. For example, studies have shown that elevated COX1 promotes memory loss and cognitive impairment after a traumatic brain injury. Traumatic brain injury (TBI), a serious health problem, is among the leading causes of acute and chronic disability in China and around the world. TBI can cause intellectual and cognitive deficits and mood and behavioral changes both short and long term (1,2). Despite the intense effort and billions of dollars invested for therapeutic measures, treatment of TBI is limited and far from satisfactory. (Shang, J. L., Cheng, Q., Yang, W. F., Zhang, M., Cui, Y., & Wang, Y. F.. (2014). Possible roles of COX-1 in learning and memory impairment induced by traumatic brain injury in mice. Brazilian Journal of Medical and Biological Research, 47(12), 1050-1056. Epub September 16, 2014; the content of which is incorporated herein by reference in its entirety).

### Inflammatory Bowel Disease

Inflammatory bowel disease (IBD) is an incurable chronic inflammatory intestinal disorder of the gastrointestinal (GI) tract that dramatically impacts quality of life. Crohn's disease (CD) and ulcerative colitis (UC) are the principal types of IBD. CD may occur in any region of the GI tract involving the ileum and colon in a discontinuous pattern by transmural inflammation, while UC affects only the colon and rectum continuously and is restricted to the mucosa. In clinical situations, CD can be associated with intestinal granulomas, strictures and fistulas, whereas these are not found in UC. In the US, IBD is the second most common inflammatory disorder and mainly affects people 15-30 years of age. The incidence and prevalence of IBD are increasing rapidly worldwide, and the situation in Asia is more severe than that in the West. Thus, IBD is becoming a major global public health problem. Multiple enzymes, such as peroxidases, NADPH oxidase (NOX), xanthine oxidase (XO), lipoxygenases (LOXs), glucose oxidase, myeloperoxidase (MPO), nitric oxide synthase (NOS), and cyclooxygenase, participate in endogenous ROS generation by catalyzing chemical reactions. (Tian Tian, Ziling Wang, Jinhua Zhang, “Pathomechanisms of Oxidative Stress in Inflammatory Bowel Disease and Potential Antioxidant Therapies”, Oxidative Medicine and Cellular Longevity, vol. 2017, Article ID 4535194, 18 pages, 2017; the content of which is incorporated herein by reference in its entirety).

Several pathways have been described for the development of IBD. For example, NF-κB activation can elevate the transcription of five types of genes that are pivotal in IBD pathogenesis: first, proinflammatory cytokines including IL-6, IL-8, IL-16, and TNF-α; second, genes modulating cell survival and propagation, such as the p53-upregulated modulator of apoptosis (PUMA), whose upregulation by NF-κB causes epithelial cell apoptosis and contributes to UC development; third, genes associated with the permeability of the intestinal barrier, for instance, the myosin light chain kinase (MYLK), which is upregulated by TNF-α-induced NF-κB activation and leads to the degradation of myosin in the intestinal barrier; fourth, metalloproteinases, which can digest mucosal cells and are also triggered by TNF-α; and fifth, enzymes including COX-2 and iNOS, which activate NF-κB and interfere with barrier stability via ROS metabolism. (Tian, 2017).

### Autoimmune Diseases

COX1 overexpression or elevated expression has been associated with the development and progression of autoimmune diseases, such as rheumatoid arthritis (RA). Several of the COX-derived PGs have been associated with the activity of RA. PGE2 is the major PG that is generated by chondrocytes and synovial fibroblasts; the biosynthesis can be enhanced by proinflammatory cytokines such as IL-1β, TNF-α, and trauma. Prostaglandin E synthase (PGES) converts COX-derived PGH2 to PGE2, a potent lipid mediator, that regulates a broad range of physiological activities in the immune and the other biological systems such as cardiovascular, endocrine, gastrointestinal, neural, pulmonary, reproductive, and visual systems. Three different forms of the PGESs have been identified, microsomal PGES-1,2 (mPGES-1,2) and cytosolic PGES(cPGES, p23). mPGES-1 is preferentially linked with COX-2 and is induced in response to various stimuli. Glutathione-independent mPGES-2 is a unique PGES that is constitutively expressed and coupled with both COXs in the production of the PGE2 involved in both tissue homeostasis and disease. The cPGES is constitutively expressed and to be preferentially coupled to COX-1 than COX-2 and its expression is not affected by proinflammatory stimuli. Of the three PGES isozymes, mPGES-1 is upregulated in synovial fluid in active RA and is minimally expressed in inactive RA. (Mohammad Javad Fattahi, Abbas Mirshafiey, “Prostaglandins and Rheumatoid Arthritis”, Arthritis, vol. 2012, Article ID 239310, 7 pages, 2012; the content of which is incorporated herein by reference in its entirety).

The mechanisms for the contribution to RA development and activity may depend on the particular PG being considered. Tissue type expression and function provide a rationale for the associated impact on joint damage, pain, immune response, and inflammation. Some studies show that prostaglandins contribute to the destruction of articular cartilage by degrading cartilage ECM, while others found that they induce chondrogenesis and terminal differentiation. (Fattahi, 2012).

COX1 inhibition is also beneficial in addressing the pathogenic antibody production, generally a characteristic of autoimmune diseases. Regulation of immune response by one or more of the COX1 derived PGs shows that abnormal production of autoantibodies is regulated or promoted by elevated COX1 activity. Therefore, inhibiting or reducing COX1 expression and thereby COX1 activity is a therapeutic option for the treatment of diseases and conditions associated with pathogenic antibody production, such as autoimmune diseases. Blaho, Victoria A et al. “Cyclooxygenase-1 orchestrates germinal center formation and antibody class-switch via regulation of IL-17.” Journal of immunology (Baltimore, Md.: 1950) vol. 183,9 (2009): 5644-53. doi:10.4049/jimmuno1.0901499; the content of which is incorporated herein by reference in its entirety).

Another study found that high levels of PGE2 and 6-keto-PGF1α (a stable metabolite of PGI2) were detected in arthritic joint tissues, correlating strongly with the intensity of synovitis. Pharmacologic inhibition of PG synthesis prevented arthritis and ameliorated active disease. (Chen, M., Boilard, E., Nigrovic, P. A., Clark, P., Xu, D., FitzGerald, G. A., Audoly, L. P. and Lee, D. M. (2008), Predominance of cyclooxygenase 1 over cyclooxygenase 2 in the generation of proinflammatory prostaglandins in autoantibody-driven K/BxN serum—transfer arthritis. Arthritis & Rheumatism, 58: 1354-1365; the content of which is incorporated herein by reference in its entirety). The importance of COX1 inhibition in RA is the impact as a disease modifier, an action beyond the abatement of symptoms of RA. COX1 inhibition reduced the downstream PGs and their downstream metabolites implicated in RA development and progression.

### Aging

Aging is usually defined as a time-dependent decline of maximal functionality that affects tissues and organs of the whole body and leads to a decreased susceptibility to disease and risk of death. Aging as a disease state is associated with several conditions often related to increased oxidative stress and cellular dysfunction.

During the aging process, cells become senescent and release proinflammatory factors that are directly and indirectly responsible for the degradation and destruction of cells and tissue throughout the body. While these proinflammatory factors may normally act as a signal for healthy function of the immune system. However, as this process accelerates in aged tissues and cells, senescent cells accumulate, causing degenerative changes perceived as ageing and age-related disease. Therefore, aging cells, are themselves in a particular disease state defined by the level or extent of which the cell or tissue is aged.

In particular, aging as a disease state has been associated with increased negative oxidative alteration on the aging heart. Studies have indicated that the basis for the age-related changes in COX is at the level of gene expression. (Jung Won Kim, Bong Sook Baek, Yun Kyung Kim, Jeremiah T. Herlihy, Yuji Ikeno, Byung Pal Yu, Hae Young Chung, Gene Expression of Cyclooxygenase in the Aging Heart, The Journals of Gerontology: Series A, Volume 56, Issue 8, 1 Aug. 2001, Pages B350—B355, https://doi.org/10.1093/gerona/56.8.B350; the content of which is incorporated herein by reference in its entirety).

Increased arachidonic acid and the enzymatic reaction with COX1 is known to play a role in promoting neuroinflammation. Aging is associated with increased inflammatory responses and vulnerability of neurons to degeneration. Some authors have raised the possibility that inflammation may occur during normal aging and increase the vulnerability to neurodegenerative disorders. (Aid, Saba, and Francesca Bosetti. “Gene expression of cyclooxygenase-1 and Ca(2+)-independent phospholipase A(2) is altered in rat hippocampus during normal aging.” Brain research bulletin vol. 73,1-3 (2007): 108-13; the content of which is incorporated herein by reference in its entirety). Additionally, COX1 upregulation with aging plays a role in altering the neuroinflammatory response and increasing vulnerability to neurodegenerative diseases with a marked inflammatory component. (Id.)

Another example of a category of diseases often associated with age are eye-disease. In particular, overexpression or upregulation of certain PGs have been associated with increased interocular pressure (TOP). TOP is known to contribute to various eye diseases and conditions, such as glaucoma. Glaucoma is a group of disorders marked by progressive degeneration and death of retinal ganglion cells (RGCs) that leads to irreversible visual decline. Globally, glaucoma is the most frequent cause of irreversible blindness (Resnikoff and Keys, 2012). Primary open-angle glaucoma is the most common form of the disease, and risk for primary open-angle glaucoma increases with age and elevated IOP (Boland and Quigley, 2007; Tham et al., 2014). Other examples of age-related eye diseases may include age-related dacular degeneration (AMD), amblyopia (lazy eye), anophthalmia and microphthalmia, astigmatism, behcet's disease of the eye, bietti's crystalline dystrophy, blepharitis, blepharospasm, cataracts, color blindness, cornea and corneal disease, diabetic eye disease, dry eye, floaters, glaucoma, healthy eyes, histoplasmosis, hyperopia (farsightedness), Idiopathic intracranial hypertension, low vision, macular edema, macular hole, macular pucker, myopia (nearsightedness), pink eye (conjunctivitis), presbyopia, refractive errors, retinal detachment, retinal disease, retinitis pigmentosa, retinoblastoma, retinopathy of prematurity, stargardt disease, usher syndrome, uveal coloboma, uveitis, vitreous detachment.

Methods for Treating Genetic Diseases Associated with Increased COX1 Activity

Some embodiments disclosed herein provide methods for treating genetic diseases associated with increased COX1 activity in a human subject in need thereof, comprising (optionally) identifying a human subject having a genetic disease and in need of an decreased expression level of a COX1 gene; and administering to the human subject an effective amount of a nitroxide antioxidant. In some embodiments, the methods disclosed herein may be used to treat a human subject that shows no symptoms of the genetic disease, but is at risk of having the genetic disease. Exemplary risk factors for genetic diseases include, but are not limited to, age, family history, health conditions, medical history, habits, or a combination thereof. In some embodiments, risk factors for genetic disease comprise an increased expression level of COX1.

In some embodiments, administering to the human subject an effective amount of the nitroxide antioxidant results in an decreased expression level of a gene, for example COX1 . The gene associated with cyclooxygenase 1 can be COX1. The treatment of the human subject with the effective amount of the nitroxide antioxidant can result in a decreased expression level of the gene. For example, the treatment can result in an decreased expression level of COX1. The decreased expression level of COX1, can decrease the quantity of the encoded protein and improve cyclooxygenase 1 activity. The improved and corrected cyclooxygenase 1 function can reduce, prevent, or eliminate the signs and symptoms of a genetic disease associated with increased COX1 function, including the curing of the genetic disease.

In some embodiments, the levels of COX1 in the connective tissue, muscle tissue, nervous tissue, and/or epithelial tissue may change after the nitroxide antioxidant is administered. Non-limiting examples of the connective tissue include dense connective tissue, loose connective tissue, reticular connective tissue, adipose tissue, cartilage, bone, and extracellular matrix. Non-limiting examples of the muscle tissue includes smooth muscle tissue, cardiac muscle tissue, and skeletal muscle tissue. Non-limiting examples of the nervous tissue include neural tissue of the central nervous system, neural tissue of the peripheral nervous system, the brain, spinal cord, cranial nerves, spinal nerves, and motor neurons. Non-limiting examples of the epithelial tissue include squamous epithelium, cuboidal epithelium, columnar epithelium, glandular epithelium, ciliated epithelium, and skin.

Non-limiting examples of genetic diseases associated with increased COX1 activity include Osteogenesis imperfecta, Spondyloepiphyseal dysplasia, Spondyloepimetaphyseal dysplasia, Achondrogenesis, hypochondrogenesis, Kniest dysplasia, Stickler syndrome, Ehlers-Danlos syndrome, Familial porencephaly, Hereditary angiopathy with nephropathy, aneurysms and muscle cramps syndrome, Benign familial haematuria, Alport syndrome, Leiomyomatosis, Bethlem myopathy, Ullrich congenital muscular dystrophy, Dystrophic epidermolysis bullosa, Corneal endothelial dystrophies Multiple epiphyseal dysplasia, Autosomal recessive Stickler syndrome, Schmid metaphyseal chondrodysplasia, Marshall syndrome, Otospondylomegaepiphyseal dysplasia Deafness, Junctional epidermolysis bullosa-other Knobloch syndrome

### Methods for Counteracting Treating a Disease Related to Aging

Some embodiments disclosed herein provide methods for counteracting age-related increase in gene expression or treating an age-related disease, comprising (optionally) identifying a human subject over the age of 35 and having an increased expression level of COX1 or an age-related disease; and administering to the human subject an effective amount of a nitroxide antioxidant. In some embodiments, the methods comprise determining the expression level of COX1. The identification step and/or the determination step may not be necessary in some instances, such as where an increased expression level of COX1 can be inferred from the human subject's age, family history, health conditions, medical history, habits, or a combination thereof. In some embodiments, the methods disclosed herein may be used to treat a human subject shows no symptoms of an age-related disease, but is at risk of having an age-related disease. Exemplary risk factors for an age-related disease include, but are not limited to, age, family history, health conditions, medical history, habits, or a combination thereof. In some embodiments, risk factors for an age-related disease comprise an increased expression level of COX1.

In some embodiments, administering to the human subject an effective amount of the nitroxide antioxidant results in an decreased expression level of a gene, for example COX1 . The gene associated with cyclooxygenase 1 can be COX1. The treatment of the human subject with the effective amount of the nitroxide antioxidant can result in a decreased expression level of the gene. For example, the treatment can result in an decreased expression level of COX1. The decreased expression level of COX1, can correct cyclooxygenase activity and prostaglandin production to a healthy level within the cell. The corrected level of cyclooxygenase activity and prostaglandin formation can result in a decrease in or disappearance of signs and symptoms of an age-related disease associated with increased COX1 function, including the curing of the age-related disease.

In some embodiments, the levels of COX1 in the connective tissue, muscle tissue, nervous tissue, and/or epithelial tissue may change after the nitroxide antioxidant is administered. Non-limiting examples of the connective tissue include dense connective tissue, loose connective tissue, reticular connective tissue, adipose tissue, cartilage, bone, and extracellular matrix. Non-limiting examples of the muscle tissue includes smooth muscle tissue, cardiac muscle tissue, and skeletal muscle tissue. Non-limiting examples of the nervous tissue include neural tissue of the central nervous system, neural tissue of the peripheral nervous system, the brain, spinal cord, cranial nerves, spinal nerves, and motor neurons. Non-limiting examples of the epithelial tissue include squamous epithelium, cuboidal epithelium, columnar epithelium, glandular epithelium, ciliated epithelium, and skin.

Some embodiments disclosed herein provide methods for treating a disease related to aging in a human subject in need thereof, comprising (optionally) identifying a human subject over the age of 35 and having an age-related disease and having an increased expression level of the COX1 gene; and administering to the human subject an effective amount of a nitroxide antioxidant. Some embodiments disclosed herein provide methods for treating an individual having or at risk of developing a condition due to aging, comprising: identifying an individual over the age of 35; and administering to the individual an effective amount of a nitroxide antioxidant, whereby the expression level of the gene associated with cyclooxygenase 1 is decreased.

Non-limiting examples of age-related diseases include cancer, rheumatoid/osteoid arthritis, systemic lupus erythematosus (SLE), inflammatory bowel disease, Alzheimer' s disease, multiple sclerosis, atherosclerosis, cardiovascular disease, cataracts, dementia, osteoporosis, type 2 diabetes, hypertension.

### Methods for Increasing Expression Level of a Gene

Some embodiments disclosed herein provide methods for increasing the expression level of a gene in a human subject in need thereof, comprising (optionally) identifying a human subject having an increased expression level of a COX1 gene ; and administering to the human subject an effective amount of a nitroxide antioxidant. Some embodiments disclosed herein provide methods for treating a disease associated with increased COX1 activity in a patient in need thereof, comprising (optionally) identifying a human subject having an increased expression level of COX1; and administering to the human subject an effective amount of a nitroxide antioxidant. The decreased expression level may be age-related, or disease related. In some embodiments, the disease may be cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, hypertension, or any combination thereof. Some embodiments disclosed herein provide methods for treating an individual in need thereof, comprising (optionally) identifying a human subject over the age of 35 in need of an decreased expression level of a COX1 gene ; and administering to the human subject an effective amount of a nitroxide antioxidant. In some embodiments, the methods comprise determining the expression level of COX1. In some embodiments, the determination step comprises inferring decreased expression level of COX1 based on the human subject's age, family history, health conditions, medical history, habits, or a combination thereof. In some embodiments, the methods disclosed herein may be used to treat a human subject shows no symptoms of a disease associated with increased COX1 function, but is at risk of having a disease associated with increased COX1 function. Exemplary risk factors for a disease associated with increased COX1 function include, but are not limited to, age, family history, health conditions, medical history, habits, or a combination thereof.

In some embodiments, administering to the human subject an effective amount of the nitroxide antioxidant results in a decreased expression level of a gene, for example a gene associated with cyclooxygenase activity . The gene associated with cyclooxygenase 1 can be COX1. The treatment of the human subject with the effective amount of the nitroxide antioxidant can result in a decreased expression level of the gene. For example, the treatment can decrease the expression levels of COX1. The decreased expression of the gene can counteract the increase in the expression level of the gene.

### Methods for Treating Cancer

Some embodiments disclosed herein provide methods for treating cancer in a human subject in need thereof, comprising (optionally) identifying a human subject having a cancer and in need of an decreased expression level of a COX1 gene; and administering to the human subject an effective amount of a nitroxide antioxidant. In some embodiments, the methods disclosed herein may be used to treat a human subject that shows no symptoms of cancer, but is at risk of having cancer. Exemplary risk factors for cancer include, but are not limited to, age, family history, health conditions, medical history, habits, or a combination thereof. In some embodiments, risk factors for cancer comprise an decreased expression level of COX1.

Non-limiting examples of the methods for identifying a human subject having a cancer include colonoscopy; sigmoidoscopy; and high-sensitivity fecal occult blood tests. In some embodiments, methods for identifying a human subject having a cancer include low-dose helical computed tomography; mammography; and pap test and human papillomavirus (HPV) testing. In some embodiments, methods for identifying a human subject having a cancer include alpha-fetoprotein blood test; breast magnetic resonance imaging (MRI); CA-125 test; clinical breast exams and regular breast self-exams; prostate-specific antigen (PSA) testing; skin exams; transvaginal ultrasound; and virtual colonoscopy. In some embodiments, methods for identifying a human subject having a cancer include barium enema; biopsy; bone marrow aspiration and biopsy; bone scan; breast MRI for early detection of breast cancer; breast MRI; colonoscopy; computed tomography (CT) scan; digital rectal exam (DRE); blood and platelets testing; bone marrow testing; umbilical cord blood testing; electrocardiogram (EKG) and echocardiogram; endoscopic techniques; fecal occult blood tests; magnetic resonance imaging (MM); mammography; multi gated acquisition (MUGA) scan; papanicolaou (pap) test; positron emission tomography and computed tomography (PET-CT) scan; sigmoidoscopy; tumor marker tests; ultrasound; upper endoscopy. In some embodiments, methods for identifying a human subject having a cancer include DNA sequencing; detecting presence of single nucleotide polymorphism (SNIP); and detecting the presence of certain protein markers.

In some embodiments, administering to the human subject an effective amount of the nitroxide antioxidant results in a decreased expression level of a gene, for example a gene associated with cyclooxygenase protein activity. The gene associated with cyclooxygenase 1 can be COX1. The treatment of the human subject with the effective amount of the nitroxide antioxidant can result in a decreased expression of the gene. For example, the treatment can result in an decreased expression level of COX1. The decreased expression level of the gene can modulate cyclooxygenase activity and prostaglandin production to a healthy rate and function. The improved cyclooxygenase activity and prostaglandin formation can result in a decrease in or disappearance of signs and symptoms of the cancer, including the curing of the cancer.

Non-limiting examples of cancer include bladder and other urothelial cancers; breast cancer; cervical cancer; colorectal cancer; endometrial cancer; endometrial cancer; esophageal cancer; liver (hepatocellular) cancer; lung cancer; neuroblastoma cancer; oral cavity and oropharyngeal cancer; ovarian, fallopian tube, and primary peritoneal cancer; prostate cancer; skin cancer; stomach (gastric) cancer; and testicular cancer.

Non-limiting examples of cancer include acute lymphoblastic leukemia, adult; acute myeloid leukemia, adult; adrenocortical carcinoma; aids-related lymphoma; anal cancer; bile duct cancer; bladder cancer; brain tumors, adult; breast cancer; breast cancer and pregnancy; breast cancer, male; carcinoid tumors, gastrointestinal; carcinoma of unknown primary; cervical cancer; chronic lymphocytic leukemia; chronic myelogenous leukemia; chronic myeloproliferative neoplasms; cns lymphoma, primary; colon cancer; endometrial cancer; esophageal cancer; extragonadal germ cell tumors; fallopian tube cancer; gallbladder cancer; gastric cancer; gastrointestinal carcinoid tumors; gastrointestinal stromal tumors; germ cell tumors, extragonadal; germ cell tumors, ovarian; gestational trophoblastic disease; hairy cell leukemia; hepatocellular (liver) cancer, adult primary; histiocytosis, langerhans cell; hodgkin lymphoma, adult; hypopharyngeal cancer; intraocular (eye) melanoma; islet cell tumors, pancreatic neuroendocrine tumors; kaposi sarcoma; kidney (renal cell) cancer; kidney (renal pelvis and ureter, transitional cell) cancer; langerhans cell histiocytosis; laryngeal cancer; leukemia, adult acute lymphoblastic; leukemia, adult acute myeloid; leukemia, chronic lymphocytic; leukemia, chronic myelogenous; leukemia, hairy cell; lip and oral cavity cancer; liver cancer, adult primary; lung cancer, non-small cell; lung cancer, small cell; lymphoma, adult Hodgkin; lymphoma, adult non-hodgkin; lymphoma, aids-related; lymphoma, primary cns; malignant mesothelioma; melanoma; melanoma, intraocular (eye); merkel cell carcinoma; metastatic squamous neck cancer with occult primary; multiple myeloma and other plasma cell neoplasms; mycosis fungoides and the sézary syndrome; myelodysplastic syndromes; myelodysplastic/myeloproliferative neoplasms; myeloproliferative neoplasms, chronic; paranasal sinus and nasal cavity cancer; nasopharyngeal cancer; neck cancer with occult primary, metastatic squamous; non-hodgkin lymphoma, adult; non-small cell lung cancer; oral cavity cancer, lip oropharyngeal cancer; ovarian epithelial cancer; ovarian germ cell tumors; ovarian low malignant potential tumors; pancreatic cancer; pancreatic neuroendocrine tumors (islet cell tumors); pheochromocytoma and paraganglioma; paranasal sinus and nasal cavity cancer; parathyroid cancer; penile cancer; pheochromocytoma and paraganglioma; pituitary tumors; plasma cell neoplasms, multiple myeloma and other; breast cancer and pregnancy; primary peritoneal cancer; prostate cancer; rectal cancer; renal cell cancer; transitional cell renal pelvis and ureter; salivary gland cancer; sarcoma, Kaposi; sarcoma, soft tissue, adult; sarcoma, uterine; mycosis fungoides and the sézary syndrome; skin cancer, melanoma; skin cancer, nonmelanoma; small cell lung cancer; small intestine cancer; stomach (gastric) cancer; testicular cancer; thymoma and thymic carcinoma; thyroid cancer; transitional cell cancer of the renal pelvis and ureter; trophoblastic disease, gestational; carcinoma of unknown primary; urethral cancer; uterine cancer, endometrial; uterine sarcoma; vaginal cancer; and vulvar cancer.

In some embodiments, non-limiting examples of cancer include, but are not limited to, hematologic and solid tumor types such as acoustic neuroma, acute leukemia, acute lymphoblastic leukemia, acute myelogenous leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer (including estrogen-receptor positive breast cancer), bronchogenic carcinoma, Burkitt's lymphoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophobalstic disease, glioblastoma, head and neck cancer, heavy chain disease, hemangioblastoma, hepatoma, hepatocellular cancer, hormone insensitive prostate cancer, leiomyosarcoma, liposarcoma, lung cancer (including small cell lung cancer and non-small cell lung cancer), lymphangioendothelio-sarcoma, lymphangiosarcoma, lymphoblastic leukemia, lymphoma (lymphoma, including diffuse large B-cell lymphoma, follicular lymphoma, Hodgkin's lymphoma and non-Hodgkin's lymphoma), malignancies and hyPerproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer (including hormone-insensitive (refractory) prostate cancer), rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), stomach cancer, squamous cell carcinoma, synovioma, sweat gland carcinoma, testicular cancer (including germ cell testicular cancer), thyroid cancer, Waldenstrom's macroglobulinemia, testicular tumors, uterine cancer, Wilms' tumor and the like.

Non-limiting examples of the cancer include acute lymphoblastic leukemia, childhood; acute myeloid leukemia/other myeloid malignancies, childhood; adrenocortical carcinoma, childhood; astrocytomas, childhood; atypical teratoid/rhabdoid tumor, childhood central nervous system; basal cell carcinoma, childhood; bladder cancer, childhood; bone, malignant fibrous histiocytoma of and osteosarcoma; brain and spinal cord tumors overview, childhood; brain stem glioma, childhood; (brain tumor), childhood astrocytomas; (brain tumor), childhood central nervous system atypical teratoid/rhabdoid tumor; (brain tumor), childhood central nervous system embryonal tumors; (brain tumor), childhood central nervous system germ cell tumors; (brain tumor), childhood craniopharyngioma; (brain tumor), childhood ependymoma; breast cancer, childhood; bronchial tumors, childhood; carcinoid tumors, childhood; carcinoma of unknown primary, childhood; cardiac (heart) tumors, childhood; central nervous system atypical teratoid/rhabdoid tumor, childhood; central nervous system embryonal tumors, childhood; central nervous system germ cell tumors, childhood; cervical cancer, childhood; chordoma, childhood; colorectal cancer, childhood; craniopharyngioma, childhood; effects, treatment for childhood cancer, late; embryonal tumors, central nervous system, childhood; ependymoma, childhood; esophageal tumors, childhood; esthesioneuroblastoma, childhood; ewing sarcoma; extracranial germ cell tumors, childhood; gastric (stomach) cancer, childhood; gastrointestinal stromal tumors, childhood; germ cell tumors, childhood central nervous system; germ cell tumors, childhood extracranial; glioma, childhood brain stem; head and neck cancer, childhood; heart tumors, childhood; hematopoietic cell transplantation, childhood; histiocytoma of bone, malignant fibrous and osteosarcoma; histiocytosis, langerhans cell; hodgkin lymphoma, childhood; kidney tumors of childhood, wilms tumor and other; langerhans cell histiocytosis; laryngeal cancer, childhood; late effects of treatment for childhood cancer; leukemia, childhood acute lymphoblastic; leukemia, childhood acute myeloid/other childhood myeloid malignancies; liver cancer, childhood; lung cancer, childhood; lymphoma, childhood Hodgkin; lymphoma, childhood non-Hodgkin; malignant fibrous histiocytoma of bone and osteosarcoma; melanoma, childhood; mesothelioma, childhood; midline tract carcinoma, childhood; multiple endocrine neoplasia, childhood; myeloid leukemia, childhood acute/other childhood myeloid malignancies; nasopharyngeal cancer, childhood; neuroblastoma, childhood; non-hodgkin lymphoma, childhood; oral cancer, childhood; osteosarcoma and malignant fibrous histiocytoma of bone; ovarian cancer, childhood; pancreatic cancer, childhood; papillomatosis, childhood; paraganglioma, childhood; pediatric supportive care; pheochromocytoma, childhood; pleuropulmonary blastoma, childhood; retinoblastoma; rhabdomyosarcoma, childhood; salivary gland cancer, childhood; sarcoma, childhood soft tissue; (sarcoma), ewing sarcoma; (sarcoma), osteosarcoma and malignant fibrous histiocytoma of bone; (sarcoma), childhood rhabdomyosarcoma; (sarcoma) childhood vascular tumors; skin cancer, childhood; spinal cord tumors overview, childhood brain and; squamous cell carcinoma (skin cancer), childhood; stomach (gastric) cancer, childhood; supportive care, pediatric; testicular cancer, childhood; thymoma and thymic carcinoma, childhood; thyroid tumors, childhood; transplantation, childhood hematopoietic; childhood carcinoma of unknown primary; unusual cancers of childhood; vaginal cancer, childhood; vascular tumors, childhood; and wilms tumor and other childhood kidney tumors.

Non-limiting examples of cancer include embryonal rhabdomyosarcoma, pediatric acute lymphoblastic leukemia, pediatric acute myelogenous leukemia, pediatric alveolar rhabdomyosarcoma, pediatric anaplastic ependymoma, pediatric anaplastic large cell lymphoma, pediatric anaplastic medulloblastoma, pediatric atypical teratoid/rhabdoid tumor of the central nervous system, pediatric biphenotypic acute leukemia, pediatric Burkitts lymphoma, pediatric cancers of Ewing's family of tumors such as primitive neuroectodermal rumors, pediatric diffuse anaplastic Wilm's tumor, pediatric favorable histology Wilm's tumor, pediatric glioblastoma, pediatric medulloblastoma, pediatric neuroblastoma, pediatric neuroblastoma-derived myelocytomatosis, pediatric pre-B-cell cancers (such as leukemia), pediatric psteosarcoma, pediatric rhabdoid kidney tumor, pediatric rhabdomyosarcoma, and pediatric T-cell cancers such as lymphoma and skin cancer.

### Methods for Treating Autoimmune Diseases

Some embodiments disclosed herein provide methods for treating an autoimmune disease in a human subject in need thereof, comprising (optionally) identifying a human subject having an autoimmune disease and in need of an decreased expression level of a COX1 gene; and administering to the human subject an effective amount of a nitroxide antioxidant. In some embodiments, the methods disclosed herein may be used to treat a human subject shows no symptoms of an autoimmune disease, but is at risk of having an autoimmune disease. Exemplary risk factors for an autoimmune disease include, but are not limited to, age, family history, health conditions, medical history, habits, or a combination thereof. In some embodiments, risk factors for an autoimmune disease comprise an increased expression level of COX1.

In some embodiments, Autoimmunity is the system of immune responses of an organism against its own healthy cells and tissues. Any disease that results from such an aberrant immune response is termed an “autoimmune disease”. Prominent examples include celiac disease, diabetes mellitus type 1, sarcoidosis, systemic lupus erythematosus (SLE), Sjögren's syndrome, eosinophilic granulomatosis with polyangiitis, Hashimoto's thyroiditis, Graves' disease, idiopathic thrombocytopenic purpura, Addison's disease, rheumatoid arthritis (RA), ankylosing spondylitis, polymyositis (PM), and dermatomyositis (DM). Autoimmune diseases are very often treated with steroids

In some embodiments, administering to the human subject an effective amount of the nitroxide antioxidant results in a decreased expression level of a gene, for example a gene associated with cyclooxygenase activity. The one or more genes associated with cyclooxygenase 1 can be COX1 or PTGS1. The treatment of the human subject with the effective amount of the nitroxide antioxidant can result in a decreased expression level of the gene. For example, the treatment can result in a decreased expression level of the one or more genes associated with cyclooxygenase 1 activity. The decreased expression levels of COX1, can decrease cyclooxygenase activity and prostaglandin formation resulting in a decrease in or disappearance of signs and symptoms of the autoimmune disease, including the curing of the autoimmune disease. In some embodiments, the decreased expression level of COX1, can improve mitochondrial function. The improved mitochondrial function can result in a decrease in or disappearance of signs and symptoms of the autoimmune disease, including the curing of the autoimmune disease.

Non-limiting examples of autoimmune diseases include rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjogren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GB S) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma). The human antibodies, and antibody portions of the present application can be used to treat humans suffering from autoimmune diseases, in particular those associated with inflammation, including, rheumatoid spondylitis, allergy, autoimmune diabetes, autoimmune uveitis.

Non-limiting examples of autoimmune diseases include acquired immunodeficiency disease syndrome (AIDS), autoimmune lymphoproliferative syndrome, hemolytic anemia, inflammatory diseases, and thrombocytopenia, acute or chronic immune disease associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-1-antitrypsin deficiency, Alzheimer's disease, amyotrophic lateral sclerosis, anemia, angina pectoris, ankylosing spondylitis associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemolytic anaemia, autoimmune hepatitis, type-1 autoimmune hepatitis (classical autoimmune or lupoid hepatitis), autoimmune mediated hypoglycaemia, autoimmune neutropaenia, autoimmune thrombocytopaenia, autoimmune thyroid disease, B cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinaemia), conjunctivitis, connective tissue disease associated interstitial lung disease, contact dermatitis, Coombs positive haemolytic anaemia, cor pulmonale, Creutzfeldt-Jakob disease, cryptogenic autoimmune hepatitis, cryptogenic fibrosing alveolitis, culture negative sepsis, cystic fibrosis, cytokine therapy associated disorders, Crohn's disease, dementia pugilistica, demyelinating diseases, dengue hemorrhagic fever, dermatitis, dermatitis scleroderma, dermatologic conditions, dermatomyositis/polymyositis associated lung disease, diabetes, diabetic arteriosclerotic disease, diabetes mellitus, Diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine, receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram negative sepsis, gram positive sepsis, granulomas due to intracellular organisms, group B streptococci (GBS) infection, Grave's disease, haemosiderosis associated lung disease, hairy cell leukemia, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpurea, Hepatitis A, Hepatitis B, Hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopaenia, idiopathic pulmonary fibrosis, idiopathic thrombocytopaenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anaemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease associated lung disease, monoclonal gammopathy, multiple myeloma, multiple systems degenerations (Mencel Dejerine-Thomas Shi-Drager and Machado-Joseph), myalgic encephalitis/Royal Free Disease, myasthenia gravis, microscopic vasculitis of the kidneys, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, Non-alcoholic Steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaceus, pemphigoid, perennial rhinitis, pericardial disease, peripheral atherlosclerotic disease, peripheral vascular disorders, peritonitis, pernicious anemia, phacogenic uveitis, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, Progressive supranucleo Palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis associated interstitial lung disease, rheumatoid spondylitis, sarcoidosis, Schmidt's syndrome, scleroderma, senile chorea, Senile Dementia of Lewy body type, sepsis syndrome, septic shock, seronegative arthropathies, shock, sickle cell anemia, Sjögren's disease associated lung disease, Sjörgren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, spondyloarthopathy, sporadic, polyglandular deficiency type I sporadic, polyglandular deficiency type II, Still's disease, streptococcal myositis, stroke, structural lesions of the cerebellum, Subacute sclerosing panencephalitis, sympathetic ophthalmia, Syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, systemic sclerosis, systemic sclerosis-associated interstitial lung disease, T-cell or FAB ALL, Takayasu's disease/arteritis, Telangiectasia, Th2 Type and Th1 Type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, yersinia and salmonella-associated arthropathy and the like.

### Nitroxide Antioxidant

Nitroxides antioxidants describes a group of stable organic molecules, containing the nitroxyl group >N—O⋅ with an unpaired electron. They have a low molecular weight, are non-toxic, do not elicit immunogenic effects on cells and easily diffuse through cell membranes. Their biological activity as antioxidants is related to the regulation of redox state in the cells. Nitroxides can undergo cyclic oxidation or reduction reactions. Their antioxidant activity is related to several mechanisms such as the direct scavenging of free radicals, transition metal ion oxidation. In addition, nitroxides exhibit superoxide dismutase (SOD)-like activity, modulate its catalase-like activity and ferroxidase-like activity, and are the inhibitors of free radical reactions such as lipid peroxidation. Nitroxides have dynamic beneficial impact on all cellular processes from inhibition of oxidative stress and reducing inflammation, while under certain conditions they may also lead to its intensification, for example, in tumor cells. The different beneficial impact on cellular processes provides each cell with necessary support to prevent or reverse diseases and conditions through optimizing cellular activity and associated biological processes in a healthy state and promoting cell death in diseases such as cancer.

Cyclic nitroxides, also known as aminoxyls or nitroxyls, are stable free radicals stabilized by methyl groups at the a position in five-membered pyrrolidine, pyrroline or oxazolidine and six-membered piperidine ring structures. The methyl groups confer stability to the nitroxide radicals by preventing radical-radical dismutation and also limit access to reactive substances, which can quench the radical species. The substituent groups on the ring (denoted by R-) produce a diverse range of compounds that can be directed to specific hydrophilic or hydrophobic regions in the cellular microenvironment. The redox transformations between the oxidation states of nitroxide, hydroxylamine and the oxoammonium cation acts as an efficient redox couple, which can support catalytic processes via reversible electron redox reactions. (Soule, Benjamin P et al. “The chemistry and biology of nitroxide compounds.” Free radical biology & medicine vol. 42,11 (2007): 1632-50. doi:10.1016/j.freeradbiomed.2007.02.030).

The mechanism of action exerted by nitroxide antioxidants is very unique. In particular, nitroxide antioxidant function is characterized by a catalytic mechanism of action associated with a single-electron redox cycle. Their reduction results in the generation of hydroxylamine and oxidation in oxoammonium ion; meanwhile both reactions are reversible and repetitive such that the ratio of free radicals suppressed by nitroxide antioxidants is significantly higher than natural antioxidant processes within a cell. Hydroxylamine also exhibits antioxidant properties because it is easily oxidized to nitroxide. As mentioned above, the nitroxides devoid of electrical charge easily diffuse through the cell membranes, thus they can also inactivate the reactive oxygen species formed in the cells and modulate the concentration of intracellular nitric oxide. Their molecular structure and composition make nitroxide antioxidants additionally efficacious in tissues that prevent transport of different molecules, such as neuronal tissue across the blood brain barrier.

Non-limiting examples of the nitroxide antioxidant include 2-ethyl-2,5,5-trimethyl-3 -oxazolidine-1-oxyl (OXANO), 2,2, 6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Amin omethyl-PROXYL, 3-Cyano-PROXYL, 3-Carbamoyl-PROXYL, 3-Carboxy-PROXYL, and 4-Oxo-TEMPO. TEMPO can also be substituted, typically in the 4 position, for example, 4-amino, 4-(2-bromoacetamido), 4-(ethoxyfluorophosphonyloxy), 4-hydroxy, 4-(2-iodoacetamido), 4-isothiocyanato, 4-maleimido, 4-(4-nitrobenzoyloxyl), 4-phosphonooxy, 2,2,6,6-tetramethyl-4-oxo-1-piperidinyloxy (TEMPONE), 1-Hydroxy-2,2,6,6-tetramethyl-4-oxo-piperidine. HCl (TEMPONE-H), 1,2-dipalmitoyl-sn-glycero-3-phospho(tempo)choline (TEMPO PC), (4-[N,N-dimethyl-N-(2-hydroxyethyl)]ammonium-2,2,6,6-tetramethylpiperidine-1-oxyl (TEMPO Choline), and the like.

The use of other nitroxide compounds is also contemplated. Nitroxide stable radicals demonstrate effective antioxidative activity in various biological systems ranging from molecular, cellular, and laboratory animal level. Nitroxides have been reported to catalyze O2. dismutation through two different catalytic pathways including reductive and oxidative reaction mechanisms. Conversely, kinetics analysis of rapid mixing stopped flow experiments de-signed to measure the effect of nitroxides on superoxide decay did not reveal any SOD activity, leading to the conclusion that nitroxides act as free radical scavengers.

Studies have shown that unlike other antioxidants, nitroxides are characterized by a catalytic mechanism of action associated with a single-electron redox cycle. Their reduction results in the generation of hydroxylamine and oxidation in oxoammonium ion; meanwhile both reactions are reversible. Hydroxylamine also exhibits antioxidant properties because it is easily oxidized to nitroxide. Nitroxide antioxidants undergo redox cycles. They are easily reduced to hydroxylamines and oxidized to oxoammonium salts.

According to certain embodiments the nitroxide compound can be selected from the following formulas:

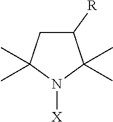

wherein X is selected from O— and OH, and R is selected from COOH, CONH, CN, and CH2NH2;

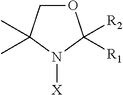

wherein X is selected from O— and OH, and R1 is selected from CH3 and spirocyclohexyl, and R2 is selected from C2H5 and spirocyclohexyl;

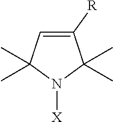

wherein X is selected from O— and OH and R is selected from CONH; and

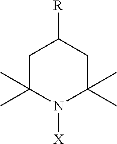

wherein X is selected from O— and OH and R is selected from H, OH, and NH2.

Suitable nitroxide compounds can also be found in Proctor, U.S. Pat. No. 5,352,442, and Mitchell et al., U.S. Pat. No. 5,462,946, both of which are hereby incorporated by reference in their entireties.

In some embodiments, the nitroxide antioxidant has a general formula:

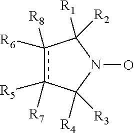

wherein the dashed line denotes a saturated bond or an unsaturated bond, wherein when the dashed line denotes an unsaturated bond, R7 and R8 are absent; R1-R4 are each independently a C1-4-alkyl, or alternatively, R1 and R2, and/or R3 and R4, together form a 3-7-membered alicyclic ring; and R5-R8 are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino,

In some embodiments, the nitroxide antioxidant includes or is associated with (e.g., binds to or is conjugated with) a bioeffector molecule. For example, the bioeffector molecule is a targeting subunit bound to the nitroxide antioxidant, such as a mitochondrial targeting subunit. A targeting subunit can direct activity of the nitroxide antioxidant to a predetermined location within or on the cell. Non-limiting examples of mitochondrial targeting bioeffector molecules includes triphenylphosphine (TPP), gramicidin, and any functional group effectively charged to be attracted to the polarized mitochondria.

In some embodiments, the nitroxide antioxidant is structurally cyclic having a ring structure including a nitroxide molecule incorporated therein. In some embodiments, the nitroxide antioxidant is characterized as the nitroxide molecule functioning as the catalytic center.

### Dosage

In some embodiments, the nitroxide antioxidant, non-toxic salts thereof, acid addition salts thereof or hydrates thereof may be administered systemically or locally, usually by oral or parenteral administration. The doses to be administered can be determined depending upon, for example, age, body weight, symptom, the desired therapeutic effect, the route of administration, and the duration of the treatment. In the human adult, the dose per person at a time can be generally from about 0.01 to about 4000 mg, by oral administration, up to several times per day. Specific examples of particular amounts contemplated via oral administration include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 or more mg. The dose per person at a time can be generally from about 0.01 to about 300 mg/kg via parenteral administration (preferably intravenous administration), from one to four or more times per day. Specific examples of particular amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more mg/kg. Continuous intravenous administration can also contemplated for from 1 to 24 hours per day to achieve a target concentration from about 0.01 mg/L to about 100 mg/L. Non-limiting examples of particular amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mg/L. The dose to be used does can depend upon various conditions, and there may be cases wherein doses lower than or greater than the ranges specified above are used.

### Compositions

The nitroxide antioxidant can be administered in the form of, for example, solid compositions, liquid compositions or other compositions for oral administration, injections, liniments or suppositories for parenteral administration.

Solid compositions for oral administration include compressed tablets, pills, capsules, dispersible powders and granules. Capsules include hard capsules and soft capsules. In such solid compositions, Tempol may be admixed with an excipient (e.g. lactose, mannitol, glucose, microcrystalline cellulose, starch), combining agents (hydroxypropyl cellulose, polyvinyl pyrrolidone or magnesium metasilicate aluminate), disintegrating agents (e.g. cellulose calcium glycolate), lubricating agents (e.g. magnesium stearate), stabilizing agents, agents to assist dissolution (e.g. glutamic acid or aspartic acid), or the like. The agents may, if desired, be coated with coating agents (e.g. sugar, gelatin, hydroxypropyl cellulose or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups and elixirs. In such compositions, the nitroxide antioxidant is dissolved, suspended or emulsified in a commonly used diluent (e.g. purified water, ethanol or mixture thereof). Furthermore, such liquid compositions may also comprise wetting agents or suspending agents, emulsifying agents, sweetening agents, flavoring agents, perfuming agents, preserving agents, buffer agents, or the like.

Injections for parenteral administration include solutions, suspensions, emulsions and solids which are dissolved or suspended. For injections, the nitroxide antioxidant can be dissolved, suspended and emulsified in a solvent. The solvents include, for example, distilled water for injection, physiological salt solution, vegetable oil, propylene glycol, polyethylene glycol, alcohol such as ethanol, or a mixture thereof. Moreover the injections can also include stabilizing agents, agents to assist dissolution (e.g. glutamic acid, aspartic acid or POLYSORBATE80™), suspending agents, emulsifying agents, soothing agents, buffer agents, preserving agents, etc. They can be sterilized in the final process or manufactured and prepared by sterile procedure. They can also be manufactured in the form of sterile solid compositions, such as a freeze-dried composition, and they may be sterilized or dissolved immediately before use in sterile distilled water for injection or some other solvent.

Other compositions for parenteral administration include liquids for external use, and ointment, endermic liniments, inhale, spray, suppositories for rectal administration and pessaries for vaginal administration which comprise the nixtroxide antioxidant and are administered by methods known in the art.

Spray compositions can comprise additional substances other than diluents: e.g. stabilizing agents (e.g. sodium sulfite hydride), isotonic buffers (e.g. sodium chloride, sodium citrate or citric acid). A small aerosol particle size useful for effective distribution of the medicament can be obtained by employing self-propelling compositions containing the drugs in micronized form dispersed in a propellant composition. Effective dispersion of the finely divided drug particles can be accomplished with the use of very small quantities of a suspending agent, present as a coating on the micronized drug particles. Evaporation of the propellant from the aerosol particles after spraying from the aerosol container leaves finely divided drug particles coated with a fine film of the suspending agent. In the micronized form, the average particle size can be less than about 5 microns. The propellant composition may employ, as the suspending agent, a fatty alcohol such as oleyl alcohol. The minimum quantity of suspending agent can be approximately 0.1 to 0.2 percent by weight of the total composition. The amount of suspending agent can be less than about 4 percent by weight of the total composition to maintain an upper particle size limit of less than 10 microns or 5 microns. Propellants that may be employed include hydrofluoroalkane propellants and chlorofluorocarbon propellants. Dry powder inhalation may also be employed.

### EXAMPLES

Some aspects of the embodiments discussed above are disclosed in further detail in the following examples, which are not in any way intended to limit the scope of the present disclosure.

In order to facilitate understanding, the specific embodiments are provided to help interpret the technical proposal, that is, these embodiments are only for illustrative purposes, but not in any way to limit the scope of the invention. Unless otherwise specified, embodiments do not indicate the specific conditions, are in accordance with the conventional conditions or the manufacturer's recommended conditions.

### Example 1

### Effects of Tempol on Expression of Genes Associated with Cyclooxygenase Activity

To assess the effects of Tempol on gene expression, Tempol was administered to experimental mice at a dose of 5 mg/g of food from 14 months to 31 months after birth. Mice receiving the same food without the addition of Tempol were used as a negative control. At the age of 31 months, the experimental animals were sacrificed and the hearts were surgically removed. The expression of a broad spectrum of genes in the cardiac tissue was assessed using chip-based microarray technology. Such chips are well known in the art and are widely used to assess gene expression. The experimental results showed that COX1 exhibited statistically significant decrease in expression. This result is shown in Table 1.

TABLE 1
Genes Associated With COX1 Exhibiting Decreased
Expression In Cardiac Tissue After Tempol Administration

| Symbol | Gene title | Fold change | P-value |
|---|---|---|---|
| PTGS1 (COX1) | Cyclooxygenase 1 | −1.18 | 0.006 |

### Example 2

### Treating Age-Related Increase in Gene Expression

A 70-kilogram human subject over the age of 65 is identified as having, or known to have, or suspected of having an increased expression level of COX1. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of COX1, is decreased.

### Example 3

### Treating a Human Subject with Decreased Gene Expression

A 70-kilogram human subject is identified as having, or known to have, or suspected of having an increased expression level of COX1. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of COX1, is decreased.

### Example 4

### Treating a Human Subject with an Age-Related Disease

A 70-kilogram human subject over the age of 65 and having a cardiovascular disease is identified for an increased expression level of COX1. Or a 70-kilogram human subject over the age of 65 is known to have a cardiovascular disease and/or decreased expression level of COX1. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of COX1, is decreased.

### Example 5

### Treating a Human Subject at Risk of Developing Cancer

A 70-kilogram human subject at risk of developing colorectal cancer is identified for increased expression level of COX1. Or a 70-kilogram human subject is known to be at risk of developing colorectal cancer and/or have increased expression level of COX1. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of COX1, is decreased.

### Example 6

### Treating a Human Subject at Risk of Developing an Autoimmune Disease

A 70-kilogram human subject at risk of developing an autoimmune disease (e.g., rheumatoid arthritis) is identified for increased expression level of COX1. Or a 70-kilogram human subject is known to be at risk of developing an autoimmune disease and/or have increased expression level of COX1. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of COX1, is decreased.

### Example 7

### Treating a Human Subject at Risk of Developing a Condition Due to Aging

A 70-kilogram human subject of 45 years old at risk of developing a condition due to aging is identified. Or a 70-kilogram human subject of 45 years old is known to be at risk of developing a condition. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of COX1, is decreased.

### Example 8

### Treating a Human Subject at Risk of Developing a Neruodegenerative Disease

A 70-kilogram human subject at risk of developing a neurodegenerative disease (e.g., Parkinson's Disease) is identified for increased expression level of COX1. Or a 70-kilogram human subject is known to be at risk of developing a neurodegenerative disease and/or have increased expression level of COX1. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of COX1, is decreased.

### Example 9

### Treating a Human Subject Having an Infection

A 70-kilogram human subject having an infection (e.g., a bacterial, fungal, or viral infection) is identified for increased expression level of COX1. Or a 70-kilogram human subject is known to have an infection and/or have increased expression level of COX1. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of COX1, is decreased.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of decreasing expression level of a gene encoding COX1, the method comprising:
identifying a human subject having a disease or condition associated with an increased expression level of the gene encoding COX1, the disease or condition selected from the group consisting of heart disease, diabetes, cancer, autoimmune disease with an elevated level of PGE2, respiratory disease mediated by one or more prostaglandins, and neuropathy mediated by one or more prostaglandins;
administering an effective amount of a nitroxide antioxidant to a subject having an increased expression level of the gene encoding COX1,
wherein the nitroxide antioxidant selected from the group consisting of 2-ethyl-2,5,5-trimethyl-3 -oxazolidine- 1-oxyl (OXANO), 2,2,6,6-tetramethylpiperidine- 1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, 4-Oxo-TEMPO, 4-amino-TEMPO, and 4-(2-bromoacetamido)-TEMPO, 4-ethoxyfluorophosphonyloxy-TEMPO, 4-hydroxy-TEMPO, 4-(2-iodoacetamido)-TEMPO, 4-isothiocyanato-TEMPO, 4-maleimido-TEMPO, 4-(4-nitrobenzoyloxyl)-TEMPO, or 4-phosphonooxy-TEMPO,
whereby the administration downregulates the expression level of the gene encoding COX1.

2. (canceled)

3. The method of claim 1, wherein downregulation of the gene treats the disease or condition.

4. (canceled)

5. The method of claim 1, wherein the disease or condition is atherosclerosis.

6. The method of claim 1, wherein the disease or condition is subject has diabetes.

7. (canceled)

8. (canceled)

9. The method of claim 1, wherein the disease or condition is cancer.

10. (canceled)

11. The method of claim 1, wherein the disease or condition is an autoimmune disease with an elevated level of PGE2.

12. The method of claim 1, wherein the nitroxide antioxidant is TEMPOL.

13. (canceled)

14. (canceled)

15. (canceled)

16. The method of claim 1, wherein the disease or condition is neuropathy mediated by one or more prostaglandins.

17. The method of claim 1, wherein the disease or condition is heart disease.

18. The method of claim 1, wherein the disease or condition is respiratory disease mediated by one or more prostaglandins.

19. (canceled)

20. (canceled)
